(19) United States
(12) Patent Application Publication
(10) Pub. No.: US 2011/0300182 A1
(43) Pub. Date: Dec. 8, 2011

(54) NUCLEIC ACID DRUG FOR TREATMENT OF ALLERGIC DISEASE

(76) Inventors: Hiroo Yokozeki, Tokyo (JP); Kazuki Hosoya, Tokyo (JP); Takahiro Satoh, Tokyo (JP); Yasuhisa Nemoto, Okinawa (JP)

(21) Appl. No.: 13/119,080

(22) PCT Filed: Mar. 12, 2009
(86) PCT No.: PCT/JP2009/055383

§ 371 Date: Jun. 1, 2011

(30) Foreign Application Priority Data
Sep. 16, 2008 (JP) 2008-237007

(51) Int. Cl. A61K 31/713; C12N 15/63; A61P 29/00; A61P 37/08; A61P 17/00; A61P 11/06; C07H 21/02; A61K 48/00

(52) U.S. Cl. 424/275.1; 536/24.5; 435/320.1

(57) ABSTRACT

This invention provides double-stranded (ds) RNA capable of suppressing the expression of STAT6 related to allergic diseases mediated by Th2-type cytokines via RNA interference and a pharmaceutical composition for prevention or treatment of allergic diseases comprising such dsRNA. Such dsRNA is a double-stranded RNA (dsRNA) molecule targeting mRNA of the STAT6 gene composed of (a) or (b): (a) a sense strand identical to a sequence having 15 to 50 continuous nucleotides in the STAT6 gene sequence represented by SEQ ID NO: 1 or 3 and an antisense strand comprising a nucleotide sequence complementary to the nucleotide sequence of the sense strand; or (b) a sense strand comprising a sequence derived from the sequence comprising 15 to 50 continuous nucleotides of the STAT6 gene sequence represented by SEQ ID NO: 1 or 3 by deletion, substitution, or addition of 1 or several nucleotides and capable of hybridizing to the STAT6 gene and an antisense strand comprising a nucleotide sequence complementary to the nucleotide sequence of the sense strand.

### TECHNICAL FIELD

The present invention relates to a double-stranded RNA (dsRNA) molecule targeting the STAT6 gene and a pharmaceutical composition used for treatment or prevention of allergic diseases comprising such dsRNA molecule.

### BACKGROUND ART

RNA interference (RNAi) is a phenomenon whereby mRNA is cleaved by double-stranded RNA (dsRNA) in a sequence-specific manner and gene expression is consequently suppressed. It is reported to be a defense mechanism at the nucleic acid level common among organisms (see Waterhouse, P. M. et al., Nature, 411: 834-843, 2001). Through RNAi, dsRNA is processed by the actions of a dicer, short interfering RNA (siRNA) is formed, siRNA functions as guide RNA for the recognition of a target sequence and the cleaving of target mRNA, and gene expression is thus suppressed.

A variety of examinations have been undertaken regarding RNAi-related technologies for the purpose of applying the same to gene function analysis via regulation of gene expression, elucidation of mechanisms of gene expression regulation, gene therapy, and the like.

Immune responses are classified into the cellular immunity mediated by the type-1 helper (Th1) cell and the humoral immunity mediated by the type-2 helper (Th2) cell. It is known that the Th1 immune responses are related to autoimmune diseases or graft rejection and the Th2 immune responses are related to allergic diseases. IL-4 and IL-13 are known as Th2-type cytokines related to Th2 immune responses.

It is reported that the STAT6 (signal transducers and activators of transcription 6) transcription regulators are important for signal transmission mediated by Th2-type cytokines (i.e., IL-4 and IL-13 receptors), and such regulators are associated with maintenance of Th2-type immune responses. It has been reported regarding STAT6 functions that STAT6-deficient mice were prepared, IL-4 information would not be transmitted to cells in such mice, and allergic reactions would not occur (see Takeda et al., Nature, Apr. 18, 1996, 380 (6575): 627-30; and Shimoda et al., Nature, Apr. 18, 1996; 380 (6575): 630-3).

### DISCLOSURE OF THE INVENTION

It is an object of the present invention to provide double-stranded (ds) RNA capable of suppressing the expression of STAT6 related to allergic diseases via RNA interference mediated by Th2-type cytokines and a pharmaceutical composition for prevention or treatment of allergic diseases containing such dsRNA.

The present inventors have conducted concentrated studies in order to attain the above object. As a result, they discovered that a dsRNA molecule comprising a sense strand having a nucleotide sequence identical to the STAT6 gene sequence or a partial sequence thereof and an antisense strand having a nucleotide sequence complementary to a nucleotide sequence of the sense strand would specifically suppress STAT6 gene expression. Further, they discovered that suppression of STAT6 gene expression would result in suppression of Th2-type cytokine or chemokine production, and such suppression would be effective for prevention and treatment of allergic diseases. This has led to the completion of the present invention.

Specifically, the present invention is as follows.

[1] A double-stranded RNA (dsRNA) molecule targeting mRNA of the STAT6 gene composed of (a) or (b) below:

(a) a sense strand identical to a sequence comprising 15 to 50 continuous nucleotides of the nucleotide sequence resulting from substitution of thymine with uracil in the STAT6 gene sequence represented by SEQ ID NO: 1 and an antisense strand comprising a nucleotide sequence complementary to the nucleotide sequence of the sense strand; or

(b) a sense strand comprising a sequence derived from the sequence comprising 15 to 50 continuous nucleotides of the nucleotide sequence resulting from substitution of thymine with uracil in the STAT6 gene sequence represented by SEQ ID NO: 1 by deletion, substitution, or addition of 1 or several nucleotides and capable of hybridizing to the STAT6 gene and an antisense strand comprising a nucleotide sequence complementary to the nucleotide sequence of the sense strand.

[2] The dsRNA molecule according to [1] composed of (c) or (d) below:

(c) a sense strand identical to a nucleotide sequence selected from the group consisting of: a sequence comprising nucleotides 3426 to 3446 of the nucleotide sequence resulting from substitution of thymine with uracil in the human STAT6 gene sequence represented by SEQ ID NO: 1; a sequence comprising nucleotides 3432 to 3452 of the nucleotide sequence resulting from substitution of thymine with uracil in the human STAT6 gene sequence represented by SEQ ID NO: 1 a sequence comprising nucleotides 259 to 279 of the nucleotide sequence resulting from substitution of thymine with uracil in the mouse STAT6 gene sequence represented by SEQ ID NO: 3; and a sequence comprising nucleotides 3026 to 3046 of the nucleotide sequence resulting from substitution of thymine with uracil in the mouse STAT6 gene sequence represented by SEQ ID NO: 3 and an antisense strand comprising a nucleotide sequence complementary to the nucleotide sequence of the sense strand; or

(d) a sense strand comprising a sequence derived from any nucleotide sequence selected from the group consisting of: a sequence comprising nucleotides 3426 to 3446 of the nucleotide sequence resulting from substitution of thymine with uracil in the human STAT6 gene sequence represented by SEQ ID NO: 1; a sequence comprising nucleotides 3432 to 3452 of the nucleotide sequence resulting from substitution of thymine with uracil in the human STAT6 gene sequence represented by SEQ ID NO: 1; a sequence comprising nucleotides 259 to 279 of the nucleotide sequence resulting from substitution of thymine with uracil in the mouse STAT6 gene sequence represented by SEQ ID NO: 3; and a sequence comprising nucleotides 3026 to 3046 of the nucleotide sequence resulting from substitution of thymine with uracil in the mouse STAT6 gene sequence represented by SEQ ID NO: 3 by deletion, substitution, or addition of 1 or several nucleotides and capable of hybridizing to the STAT6 gene sequence and an antisense strand comprising a nucleotide sequence complementary to the nucleotide sequence of the sense strand.

[3] The dsRNA molecule according to [2], which is composed of any base pairs selected from the group consisting of: base pairs comprising a sense strand represented by SEQ ID NO: 5 and an antisense strand represented by SEQ ID NO: 6; base pairs comprising a sense strand represented by SEQ ID NO: 7 and an antisense strand represented by SEQ ID NO: 8; base pairs comprising a sense strand represented by SEQ ID NO: 9 and an antisense strand represented by SEQ ID NO: 10; base pairs comprising a sense strand represented by SEQ ID NO: 11 and an antisense strand represented by SEQ ID NO: 12; and base pairs comprising a sense strand represented by SEQ ID NO: 13 and an antisense strand represented by SEQ ID NO: 14.

[4] The dsRNA molecule according to any of [1] to [3], wherein the sense strand is ligated to the antisense strand via a linker molecule.

[5] A vector comprising template DNA of the dsRNA molecule according to [4] and expressing the dsRNA molecule.

[6] A pharmaceutical composition used for treatment or prevention of allergic diseases comprising 1 or a plurality of the dsRNA molecule(s) according to any of [1] to [4] and capable of suppressing STAT6 gene expression.

[7] A pharmaceutical composition used for treatment or prevention of allergic diseases comprising the vector according to [5] and capable of suppressing STAT6 gene expression.

[8] The dsRNA molecule according to any of [1] to [3], which has an overhanging nucleotide comprising one or a plurality of guanines (Gs) at the 5′ end of the sense strand.

[9] The dsRNA molecule according to [8], wherein the sense strand is ligated to the antisense strand via a linker molecule.

[10] A vector comprising template DNA of the dsRNA molecule according to [9] and expressing the dsRNA molecule.

[11] A pharmaceutical composition used for treatment or prevention of allergic diseases comprising 1 or a plurality of the dsRNA molecule(s) according to [8] or [9], which induces no interferon and/or cell damage and is capable of suppressing STAT6 gene expression.

[12] A pharmaceutical composition used for treatment or prevention of allergic diseases comprising the vector according to [10], which induces no interferon and/or cell damage and is capable of suppressing STAT6 gene expression.

[13] The pharmaceutical composition used for treatment or prevention of allergic diseases according to [6], [7], [11], or [12], which is administered through the nasal cavity.

[14] The pharmaceutical composition used for treatment or prevention of allergic diseases according to [6], [7], [11], or [12], which is an ointment to be applied to the skin.

This description includes part or all of the contents as disclosed in the description and/or drawings of Japanese Patent Application No. 2008-237007, which is a priority document of the present application.

### BRIEF DESCRIPTION OF THE DRAWINGS

FIG. 1 is a schematic diagram showing the experimental protocols 1 (A) and 2 (B) of the examples.

FIG. 2 shows the structure of siRNA and that of shRNA.

FIG. 3 shows the suppression of STAT6 expression via introduction of human STAT6 siRNA.

FIG. 4 shows eotaxin-3 production by human fibroblasts into which human STAT6 siRNA has been introduced.

FIG. 5 shows the suppression of STAT6 expression at the mRNA level in human fibroblasts into which human STAT6 shRNA has been introduced.

FIG. 6 shows the suppression of STAT6 expression at the protein level in human fibroblasts into which human STAT6 shRNA has been introduced.

FIG. 7 shows eotaxin-3 production by human fibroblasts into which human STAT6 shRNA has been introduced.

FIG. 8 shows the results of the suppression of STAT6 expression in normal fibroblasts via introduction of mouse STAT6 siRNA, confirmed via Western blotting.

FIG. 9 shows the results of the suppression of STAT6 expression in normal fibroblasts via introduction of mouse STAT6 siRNA, confirmed via RT-PCR.

FIG. 10 shows the results of quantification via ELISA of the concentration of eotaxin (CCL11) produced by IL-4 and TNF-alpha costimulation in normal fibroblasts via introduction of mouse STAT6 siRNA.

FIG. 11 shows the effects of STAT6 siRNA on contact hypersensitivity responses. FIG. 11A shows the effects of TNCB on contact hypersensitivity responses, FIG. 11B shows the effects of DNFB on contact hypersensitivity responses, and FIG. 11C shows the effects of oxazolone on contact hypersensitivity responses.

FIG. 12 shows the effects of STAT6 siRNA on contact hypersensitivity responses via Giemsa staining.

FIG. 13 shows the effects of STAT6 siRNA on contact hypersensitivity responses using profiles of infiltrated cells.

FIG. 14 shows the effects of a STAT6 siRNA-containing ointment on contact hypersensitivity responses.

FIG. 15 shows the effects of STAT6 siRNA on rhinitis models based on the number of times of sneezing.

FIG. 16 shows the effects of STAT6 siRNA on rhinitis models via Giemsa staining.

FIG. 17 shows the effects of STAT6 siRNA on rhinitis models based on the number of infiltrated eosinophils.

### BEST MODES FOR CARRYING OUT THE INVENTION

Hereafter, the present invention is described in detail.

The present invention relates to a double-stranded RNA (dsRNA) molecule targeting mRNA of the STAT6 (signal transducers and activators of transcription 6) transcription regulator. A strand of the dsRNA molecule is a sense strand having a nucleotide sequence identical to the gene sequence of the STAT6 transcriptional factor or a partial sequence thereof and capable of hybridizing thereto. Another strand is an antisense strand comprising a nucleotide sequence complementary to the nucleotide sequence of the sense strand. The sense strand complementarily binds to the antisense strand. In such a case, it is not necessary that the sense strand and the antisense strand are completely complementary to each other. As long as such sequences complementarily bind, 1 or a plurality of mismatches; i.e., 1 to 10, preferably 1 to 5, more preferably 1 to 3, and 2 or 1 mismatches may be present. A target sequence in the STAT6 gene may be a coding or non-coding region.

In the present invention, the term “dsRNA molecule” refers to the siRNA molecule and the shRNA molecule. In the present invention, the dsRNA molecule is capable of forming the miRNA molecule.

The number of nucleotides in the target sequence of the STAT6 gene of the dsRNA molecule of the present invention is not particularly limited. It is 15 to 50, 15 to 45, 15 to 40, 15 to 35, or 15 to 30 nucleotides, preferably 20 to 35 nucleotides, more preferably 21 to 35, 21 to 25, or 21 to 23 nucleotides, and particularly preferably 21 nucleotides.

In the present invention, the term “a sense strand having a nucleotide sequence identical to the STAT6 gene sequence or a partial sequence thereof and capable of hybridizing thereto” refers to an RNA sequence having the nucleotide sequence resulting from substitution of thymine with uracil in the human STAT6 gene sequence represented by SEQ ID NO: 1 or the mouse STAT6 gene sequence represented by SEQ ID NO: 3. A sense strand constituting the dsRNA molecule of the present invention is preferably identical to the STAT6 gene sequence, and a substantially identical sequence may be sufficient. As long as the sense strand of the dsRNA molecule hybridizes to the actual target STAT6 mRNA sequence, specifically, a mismatch may occur via deletion, substitution, or addition of 1 or a plurality of nucleotides (i.e., 1 to 10, preferably 1 to 5, more preferably 1 to 3, or 2 or 1 nucleotides). In such a case, hybridization is carried out under in vivo conditions when the dsRNA of the present invention is used in the form of a pharmaceutical agent via administration thereof to an organism. When dsRNA of the present invention is used in the form of a reagent in vitro, hybridization is carried out under moderately to highly stringent conditions. An example thereof is a condition in which hybridization is carried out in the presence of 400 mM NaCl, 40 mM PIPES (pH: 6.4), and 1 mM EDTA at 50° C. to 70° C. for 12 to 16 hours.

The sense strand of the dsRNA of the present invention has 90% or higher, preferably 95% or higher, and more preferably 96, 97, 98, or 99% or higher sequence identity to the target sequence, which is determined with the use of default parameters (i.e., the initially set parameters) via a homology search program known to a person skilled in the art, such as BLAST (J. Mol. Biol., 215, 403-410, 1990) or FASTA (Methods Enzymol., 183, 63-98, 1990).

The dsRNA molecule of the present invention can be provided in the form of a short hairpin RNA (shRNA) molecule in which the sense strand and the antisense strand defined above are ligated to each other via a linker molecule, and a loop structure is formed and folded at the linker site. The shRNA molecule is processed by a dicer and it is capable of forming a siRNA molecule. As long as a linker molecule contained in the shRNA molecule is capable of ligating a sense strand to an antisense strand to form a stem-loop structure, a polynucleotide linker or non-polynucleotide linker may be used. A polynucleotide linker known to a person skilled in the art is preferable, although it is not limited thereto. A hairpin-loop sequence is not limited. An example thereof is a sequence comprising 5 to 12 nucleotides and beginning with UU (e.g., UUCAAGAGA). Loop sequences described in, for example, Lee N S. et al., 2002, Nat. Biotech., 20, 500-505, Paddison P. J. et al., 2002, Genes and Dev. 16, 948-958, Sui G. et al., 2002, Proc. Natl. Acad. Sci. USA 99, 5515-5520, Paul C P. et al., 2002, Nat. Biotech. 20, 505-508, and Kawasaki H. et al., 2003, Nucleic Acids Res. 31, 700-707, can be adopted. The shRNA molecule can be synthesized in vitro or in vivo in accordance with known techniques as described above. When synthesizing the shRNA molecule, a single RNA strand comprising a sense strand and an inverted antisense strand is synthesized, and the single RNA strand is converted into a double-strand via self-complementary binding. Thus, the shRNA molecule can be obtained.

The sense or antisense strand constituting the dsRNA molecule may comprise an overhang at the 3′ end according to need. The type and the number of nucleotides constituting such overhang are not limited. For example, a sequence comprises 1 to 5, preferably 1 to 3, and more preferably 1 or 2 nucleotides. Examples of such sequence include TTT, UU, and TT. In the present invention, the term “overhang” refers to a nucleotide added to the end of a strand of the dsRNA molecule that does not have a nucleotide at a corresponding site of another strand to which the overhang can complementarily bind. An overhang may comprise nucleotides that naturally constitute DNA of the target STAT6 gene. For example, the dsRNA of the present invention is composed of a sense strand comprising 21 continuous nucleotides in the nucleotide sequence resulting from substitution of thymine with uracil in the STAT6 gene sequence, an antisense strand having a sequence complementary to a nucleotide sequence lacking 2 nucleotides at the 3′ end of the sense strand, and an overhang of two nucleotides at the 3′ end of the sense or antisense strand.

Also, the dsRNA of the present invention may have at the 5′ end of the sense strand an overhang comprising 1 to 3, and preferably 3, 2, or 1 guanine(s) (Gs). By providing an overhang comprising 1 or a plurality of Gs at the 5′ end of the sense strand, interferon expression would not be induced in cells into which shRNA had been introduced (Gondai et al., Nucleic Acids Research, 2008, Vol. 36, No. 3, e18, Epub., Jan. 21, 2008).

In addition, the sense or antisense strand constituting the dsRNA molecule may comprise substitution, addition, or deletion of 1 to 3 nucleotides, and preferably 1 or 2 nucleotides, according to need, in order to smoothly carry out various experimental operations, such as gene sequencing, provided that siRNA would not be influenced.

The 5′ end of the sense or antisense strand may be phosphorylated according to need. Triphosphoric acid (ppp) may bind to the 5′ end of the shRNA molecule. Triphosphoric acid (ppp) may bind to the overhang comprising G at the 5′ end of the sense strand as described above.

In the present invention, a sense strand is preferably identical to a nucleotide sequence selected from the group consisting of: a sequence comprising nucleotides 3426 to 3446 of the nucleotide sequence resulting from substitution of thymine with uracil in the human STAT6 gene sequence represented by SEQ ID NO: 1; a sequence comprising nucleotides 3432 to 3452 of the nucleotide sequence resulting from substitution of thymine with uracil in the human STAT6 gene sequence represented by SEQ ID NO: 1; a sequence comprising nucleotides 259 to 279 of the nucleotide sequence resulting from substitution of thymine with uracil in the mouse STAT6 gene sequence represented by SEQ ID NO: 3; and a sequence comprising nucleotides 3026 to 3046 of the nucleotide sequence resulting from substitution of thymine with uracil in the mouse STAT6 gene sequence represented by SEQ ID NO: 3. It is particularly preferable that a sense strand be identical to a sequence comprising nucleotides 3426 to 3446 of the nucleotide sequence resulting from substitution of thymine with uracil in the human STAT6 gene sequence represented by SEQ ID NO: 1 or a sequence having nucleotides 3432 to 3452 of the nucleotide sequence resulting from substitution of thymine with uracil in the human STAT6 gene sequence represented by SEQ ID NO: 1.

The dsRNA molecule of the present invention comprises base pairs selected from the group consisting of: base pairs comprising a sense strand represented by SEQ ID NO: 5 and an antisense strand represented by SEQ ID NO: 6; base pairs comprising a sense strand represented by SEQ ID NO: 7 and an antisense strand represented by SEQ ID NO: 8; base pairs comprising a sense strand represented by SEQ ID NO: 9 and an antisense strand represented by SEQ ID NO: 10; base pairs comprising a sense strand represented by SEQ ID NO: 11 and an antisense strand represented by SEQ ID NO: 12; and base pairs comprising a sense strand represented by SEQ ID NO: 13 and an antisense strand represented by SEQ ID NO: 14. A dsRNA molecule composed of a sense strand represented by SEQ ID NO: 5 and an antisense strand represented by SEQ ID NO: 6 or a sense strand represented by SEQ ID NO: 7 and an antisense strand represented by SEQ ID NO: 8 is particularly preferable.

The dsRNA of the present invention can be chemically synthesized or synthesized in vitro via a transcription system using a promoter and RNA polymerase. In the case of chemical synthesis, single-stranded RNA having inverted complementary sequences and having self-complementarity may be synthesized, and such sequences may be bound to each other at the self-complementarity region. The synthesized sense and antisense strands can be annealed by a common method known to a person skilled in the art. The synthesized sense and antisense strands are dissolved in a buffer for dsRNA annealing, equal amounts (equimolar numbers) thereof are mixed, temperature is raised until double strands are dissociated from each other, and the resultants are incubated via gradual cooling. Thus, dsRNA annealing can be carried out. Annealing may be carried out by allowing the dsRNA to stand at 90° C. for 1 minute and then at 37° C. for 1 hour, for example. Thereafter, phenol/chloroform extraction and ethanol precipitation may be carried out to obtain the dsRNA molecule. Synthesis using a promoter and RNA polymerase may be carried out by synthesizing template DNA having a promoter and, at a site downstream thereof, a sense strand ligated to an antisense strand to form a loop structure and transcribing RNA with the aid of RNA polymerase. In order to add an overhang sequence of G to the 5′ end of the sense strand, a sequence comprising G may be added to the end of a promoter. In this case, a DNA sequence adequately comprises a regulator sequence, such as a terminator. Promoter and/or other regulator sequences are functionally linked to a vector. The term “functionally linked” used herein refers to a situation in which promoter and/or other regulator sequences are linked and incorporated into a vector, so that the dsRNA molecule is expressed and target STAT6 mRNA is degraded in cells into which the vector has been introduced under the control of the promoter and/or other regulator sequences. As a terminator, for example, a TTTTTT sequence may be used. As a promoter, a constitutive promoter, a tissue-specific promoter, a stage-specific promoter, or the like can be used. In the case of in vitro production, T3 promoter, T7 promoter, or the like may be used. When template DNA of double-stranded RNA of the present invention is introduced into a vector and such vector is administered to an organism to synthesize double-stranded RNA in vivo, PolIII promoters, such as U6 promoter and H1 promoter, are used. When a vector is used, a plasmid, virus, or other vector can be used. pBAsi, pSUPER, and other vectors may be used as plasmid vectors, and adenovirus, lentivirus, retrovirus, and other vectors may be used as virus vectors. When T7 promoter or the like is used for synthesizing the dsRNA of the present invention, the existence of a sequence of G activates T7 promoter, and the efficiency for dsRNA production is advantageously enhanced.

The present invention also includes a vector that expresses the dsRNA molecule described above.

The dsRNA molecule of the present invention is capable of cleaving STAT6 mRNA in a sequence-specific manner, inducing RNA interference (RNAi) that suppresses STAT6 gene expression, and consequently knocking down the STAT6 gene. As a result of STAT6 gene knockdown, production of Th2 cytokine and chemokine is suppressed. For example, infiltration of mononuclear cells, eosinophils, neutrophils, mast cells, lymphocytes, and the like is suppressed at inflammatory sites, such as skins, and inflammation is suppressed.

When the dsRNA molecule of the present invention is provided in the length of 30 nucleotides or longer, it is processed by the action of an RNaseIII-like enzyme that is referred to as a dicer, and a small interfering RNA (siRNA) molecule comprising 21 to 27 nucleotides having an overhang of 2 nucleotides at the 3′ end can be formed. Such siRNA molecule is incorporated into a protein complex referred to as the RNA-induced silencing complex (RISC), and it recognizes and degrades STAT6 mRNA based on its homology to the siRNA molecule to suppress STAT6 gene expression.

The dsRNA molecule of the present invention can be used for treatment and prevention of allergic diseases. Examples of allergic diseases include rhinostenosis, allergic bronchitis, allergic conjunctivitis, inflammatory disease, rash, hives, atopic dermatitis, allergic rhinitis (pollinosis), allergic conjunctivitis, allergic gastroenteritis, bronchial asthma, pediatric asthma, alimentary allergy, drug allergy, and inflammatory diseases.

The present invention includes a pharmaceutical composition used for treatment or prevention of allergic diseases comprising the dsRNA molecule targeting the STAT6 gene. Such composition may comprise 1 or a plurality of types of the dsRNA molecules of the present invention or vectors in combination. For example, the composition may comprise 1, 2, or 3 types of dsRNA molecules selected from the group consisting of: a dsRNA molecule comprising a sense strand represented by SEQ ID NO: 5 and an antisense strand represented by SEQ ID NO: 6; a dsRNA molecule comprising a sense strand represented by SEQ ID NO: 7 and an antisense strand represented by SEQ ID NO: 8; a dsRNA molecule comprising a sense strand represented by SEQ ID NO: 9 and an antisense strand represented by SEQ ID NO: 10; a dsRNA molecule comprising a sense strand represented by SEQ ID NO: 11 and an antisense strand represented by SEQ ID NO: 12; and a dsRNA molecule comprising a sense strand represented by SEQ ID NO: 13 and an antisense strand represented by SEQ ID NO: 14.

When analytes are cells or tissue, the dsRNA of the present invention can be introduced by culturing the dsRNA simultaneously with such cells or tissue. Also, a method involving the use of calcium ions, electroporation, the spheroplast method, the lithium acetate method, the calcium phosphate method, lipofection, microinjection, or other methods may be employed to introduce dsRNA. When an analyte is an individual animal, dsRNA can be administered through an oral route, a nasal route, intravenous, intramuscular, subcutaneous, or intraperitoneal injection, or a non-enteral route. In order to treat asthma, rhinitis, or other diseases, dsRNA can be administered in the form of a liquid or aerosol mixture with a known penetrating agent. In order to treat dermatitis or the like, it can be administered topically by applying the same in the form of an ointment to the skin lesion. Such ointment comprises, as a carrier, fat, fatty oil, lanolin, petroleum jelly, paraffin, wax, plaster, resin, plastic, glycols, higher alcohol, glycerin, water or an emulsifier, or a suspending agent. In such a case, dsRNA may be administered in the form of a mixture with a positively charged cationic polymer. Examples of cationic polymers include polyethyleneimine (PEI: linear or branched), polyamine, and commercially available gene transfer reagents. Examples of commercially available gene transfer reagents include Lipofectamine®, Lipofectamine 2000, and Lipofectamine RNAiMAX.

When dsRNA is administered to a specific site, it may be delivered to such site with the utilization of a drug delivery system. A variety of known drug delivery systems are available, and an adequate means can be adopted in accordance with a site of interest. Examples of drug delivery systems include known methods utilizing liposomes, emulsions, and polylactic acids as carriers. It is preferable that dsRNA be administered in the form of a mixture with a pharmaceutically acceptable diluent or carrier. Examples of adequate carriers include, but are not limited to, physiological saline, phosphate buffered saline, phosphate buffered saline in a glucose solution, and buffered saline. The amount of dsRNA to be introduced can be adequately determined in accordance with, for example, a type of disease to be prevented or treated, severity thereof, and age, body weight, and other conditions of a subject. It is preferable that at least a copy of dsRNA be introduced per cell in a lesion area. For example, 1 nM to 100 μM, preferably 10 nM to 50 μM, and more preferably 100 nM to 20 μM dsRNA molecules are administered per dose.

According to the present invention, the condition in which STAT6 gene expression is suppressed (silenced) via RNA interference refers to a situation in which such gene expression is suppressed by 75% or more, 50% or more, or 20% or more, as well as 100%, when the STAT6 gene expression level is determined using the gene expression level at the mRNA or protein level as the indicator, compared with the case in which the dsRNA of the present invention is not introduced. The degree of expression suppression may be determined by comparing the amounts of mRNA or protein production of the STAT6 gene before and after the introduction of dsRNA. In the case of mRNA, the degree of expression suppression can be determined via Northern hybridization, RT-PCR, in situ hybridization, or other means. In the case of proteins, such degree can be determined via Western blotting, ELISA, assay involving the use of a protein chip to which antibodies have been bound, protein activity assay, or other means. The dsRNA of the present invention having at the 5′ end of the sense strand an overhang comprising 1 or several Gs would not induce interferon reactions in the cells or organisms into which such dsRNA has been introduced. The situation in which it would not induce interferon reactions refers to a situation in which interferon α or β expression is not induced. It also refers to not only a situation in which interferon synthesis would not be activated but also a situation in which a pathway associated with interferon would not be activated. A case in which interferon expression is suppressed by 75% or more, 50% or more, 20% or more, or 10% or more is within the scope of the present invention as well as a case in which it is suppressed completely. Whether or not the interferon reaction has occurred can be determined by assaying the production of interferon or interferon mRNA. Such assay may be carried out via Northern hybridization, RT-PCR, in situ hybridization, Western blotting, ELISA, assay involving the use of a protein chip to which antibodies have been bound, protein activity assay, or other means as described above.

The dsRNA of the present invention would not cause cell damage to cells or cells of an organism even if it is introduced thereinto. When double-stranded RNA is administered, in general, cell damage would be induced via activation of dsRNA-dependent protein kinase (PKR). However, the dsRNA of the present invention would not activate PKR or cause cell damage. The term “cell damage” used herein refers to a situation in which cells are damaged to the extent that normal functions are not exerted or growth is suppressed. The term also refers to a cell death, such as apoptosis or necrosis. In the present invention, the term “ . . . would not cause cell damage” refers not only a situation in which cell damage would not be induced at all but also a situation in which the number of cells experiencing cell damage is 75% or less, 50% or less, 20% or less, or 10% or less than the number of such cells when double-stranded RNA comprising a sense strand without a sequence of G at the 5′ end is introduced. Whether or not cell damage is induced can be determined by observing cells and inspecting the development of the cytopathic effects (CPE). Also, it can be determined by assaying the metabolic activity of cells or a dye-exclusion test, such as trypan blue-exclusion. The dsRNA of the present invention would not induce both or either of interferon and cell damage. In the present invention, the situation in which “inducing no interferon and/or cell damage” refers to not only a situation in which induction is completely inhibited but also a situation in which induction is reduced. The degree of inhibition of interferon and/or cell damage induction may vary depending on a test system, an analyte, and other conditions. dsRNA that does not induce interferon and/or cell damage in at least 1 system or analyte is within the scope of the dsRNA of the present invention that does not induce interferon and/or cell damage.

Further, the present invention includes a method of administering the dsRNA of the present invention to an organism and suppressing STAT6 gene expression in the organism without inducing interferon expression and a method of administering the dsRNA of the present invention to an animal in order to prevent or treat allergic diseases associated with the STAT6 gene without inducing interferon expression.

The present invention is described in detail with reference to the following examples, although the present invention is not limited to these examples.

### Method

### Animal

BALB/c mice were purchased from Sankyo Labo Service Corporation. All mice were raised by supplying feeds and water at the sterile facilities in accordance with the Guidelines for Animal Experimentation of Tokyo Medical and Dental University. 7- to 12-week-old mice were subjected to the experiment, and a group consisted of at least 4 mice.

Preparation of Human STAT6 Small Interfering RNA (siRNA) and Small Hairpin RNA (shRNA) and Evaluation of Effects Thereof

Small RNAs to be subjected to the experiments (i.e., STAT6 siRNA and STAT6 shRNA) were prepared by Tokyo Medical and Dental University in collaboration with HaploPharma Inc. Based on the theory leading to more potent RNAi than existing RNAi (Ui-Tei K, et al., Nucleic Acids Res., 2004, 32: 936-948), siRNA candidate sequences were first determined by using the BLAST Search (http://www.ncbi.nml.nih,gov/blast/index.shtml; avoidance of off-target effects) and predicting the mRNA higher-order structure (http://www.bioinfo.rpi.edu/˜zukerm/).

Six candidate sequences were selected, and the effects thereof were evaluated by introducing such sequences into normal human dermal fibroblasts sampled from human skin tissue biopsy specimens. After siRNA of the sequence exhibiting efficient RNA interference was selected, shRNA exhibiting substantially no non-specific reactions (interferon responses) at the time of introduction was prepared based on such sequence, and the RNAi effects thereof were also examined.

Selection of Mouse STAT6 siRNA Candidate

Since use of mice was planned for the in vivo experiment, it was necessary to examine mouse STAT6 siRNA in vitro as in the case of human STAT6 siRNA. Based on the theory same as that for human STAT6 siRNA, 3 types of candidate sequences for mouse STAT6 siRNA were designed. The effects of RNA interference were evaluated using normal fibroblasts sampled from the dorsal skin of newborn mice.

### Cell Culture and Stimulation

STAT6 siRNA was introduced into cells using Lipofectamine™ 2000 (Invitrogen).

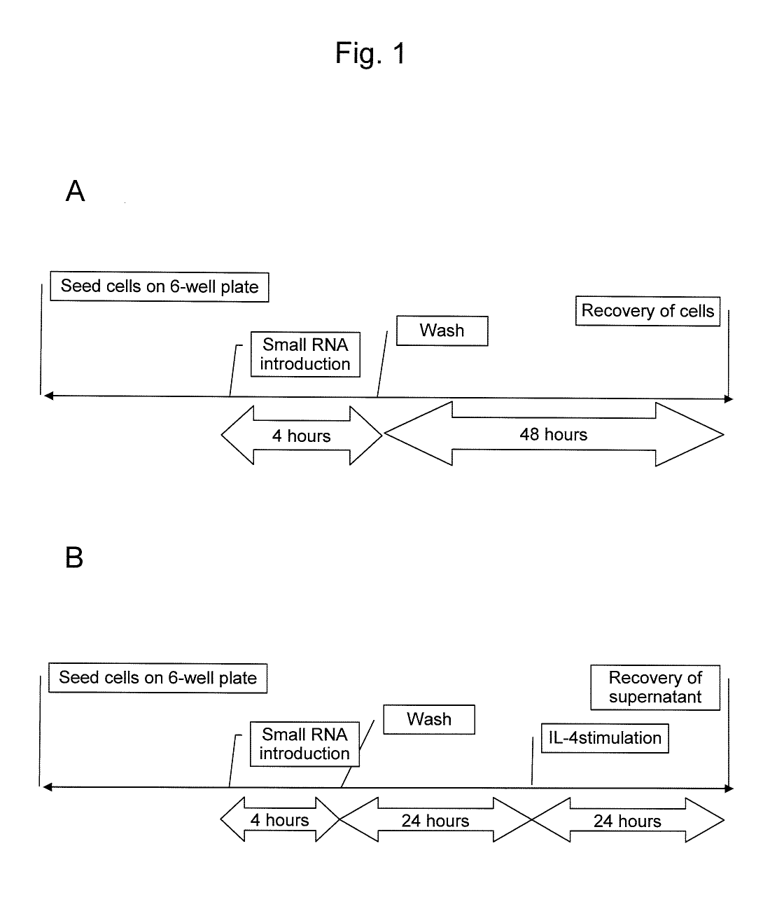

STAT6 siRNA was introduced into cells based on two types of protocols. The protocols are shown in FIG. 1A and FIG. 1B.

### Experimental Protocol 1

Normal fibroblasts are seeded on a 6-well plate. Culture is conducted using a culture solution prepared by adding 10% fetal calf serum (FCS, Sigma) and 1% antibiotics/antimycotics (Gibco-BRL) to the Dullbeco's modified Eagle's medium (DMEM, Sigma) at 37° C. in the presence of 5% CO₂. siRNA or shRNA is introduced under 60% to 70% subconfluent conditions, culture is continued for an additional 48 hours, and cells are then recovered.

### Experimental Protocol 2

Introduction of siRNA or shRNA is carried out in the same manner as in Protocol 1. After siRNA or shRNA is introduced, the culture solution is replaced with another culture solution prepared by adding 2% FCS and 1% antibiotics/antimycotics to DMEM (serum starvation). Cells were stimulated with rhIL-4 (with 10 ng/ml rmIL-4 and 40 ng/ml TNFα in the case of mouse, R & D) 24 hours later, and the cell culture supernatant is recovered 24 hours thereafter.

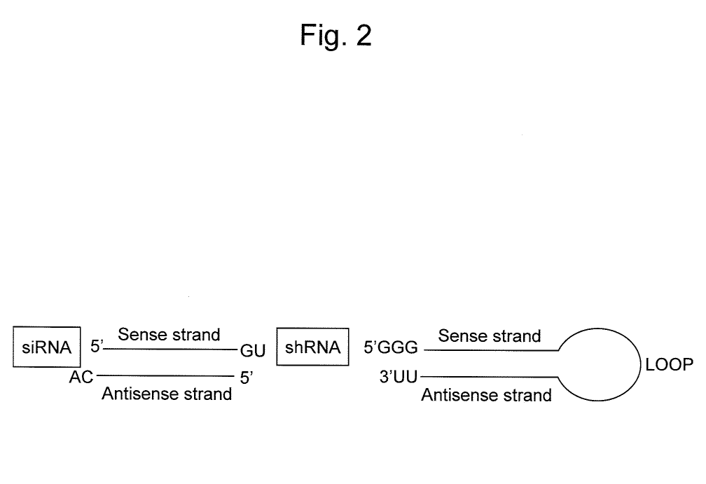

Since shRNA is introduced into cells before it is degraded by a dicer, shRNA functions more specifically than siRNA that separately expresses double strands, and it is capable of reducing the interferon responses caused by introduction. FIG. 2 shows the structure of shRNA and that of siRNA.

### Induction of Acute Contact Hypersensitivity Responses by Hapten

Mice were sensitized by applying 50 μl of 5% 2,4,6-trinitrochlorobenzene (TNCB) (in acetone:olive oil=4:1) (Nacalai Tesque), 0.5% 2,4-dinitrofluorobenzene (DNFB) (in acetone: olive oil=4:1) (Nacalai Tesque), or 5% 4-ethoxymethylene-2-phenyl-2-oxazolin-5-one (oxazolone) (in acetone: olive oil=4:1) (Sigma) to the shaved abdominal region on Day 0 (DNFB was applied continuously on Days 0 and 1). Thereafter, 20 μl of 1% TNCB (in acetone:olive oil=1:4), 0.2% DNFB (in acetone:olive oil=4:1), or 1% oxazolone (in acetone:olive oil=4:1) was applied to the auricles on Day 5 to elicit the response. Auricular swelling was measured using a dial thickness gauge (Peacock) as the indicator for the contact hypersensitivity responses.

### Western Blotting

Proteins were extracted from cells using a cell lysis buffer (Cell Signaling). After electrophoresis was carried out, the product was transferred to Hybond-P (Amersham Pharmacia Biotech). Electrophoresis was carried out at 80 V for 30 minutes and then at 130 V for 60 minutes. The reaction was allowed to proceed with the use of anti-human β-actin antibodies or anti-mouse β-actin antibodies and anti-human STAT6 antibodies or anti-mouse STAT6 antibodies (Santa Cruz Biotechnology) as the primary antibodies, and the reaction was then carried out with the use of peroxidase-labeled anti-rabbit immunoglobulins antibodies (DAKO) as the secondary antibodies. Color development was carried out using the ECL plus Western blotting detection reagents (GE Healthcare).

### RNA Extraction and Reverse Transcription

RNA was extracted from normal fibroblasts using ISOGEN (Nippon Gene Co., Ltd.). Reverse transcription was carried out using a reaction buffer containing a hexanucleotide mixture (A260, 6.25 U/ml, Roche), dNTPs (0.125 mM, Takara Bio), the human placenta RNase inhibitor (80 U, Takara Bio), and a reverse transcriptase (400 U, Moloney murine leukemia virus, Takara Bio) and 800 ng of RNA. The total amount of the reaction solution was adjusted to 40 μl, and the reaction was allowed to proceed at 37° C. for 60 minutes.

### Quantitative Polymerase Chain Reaction (PCR)

Quantitative PCR was carried out using the Brilliant SYBR Green QPCR Master Mix (Stratagene) in accordance with the protocols. Measurement was carried out using the Mx3000P Real-Time PCR system (Stratagene).

Administration of STAT6 siRNA to Mice

On Day 3, 30 μl of STAT6 siRNA (in OPTI-MEM® I:Lipofectamine™ 2000=50:1) was administered subcutaneously to the auricles (1 nmol/ear).

STAT6 siRNA-Containing Ointment

The STAT6 siRNA-containing ointment was prepared using hydrophilic petrolatum as a base to adjust the concentration of STAT6 siRNA to 2%. The resulting ointment was applied to the auricles on Day 3 in an amount of 10 nmol/ear.

### Preparation of Rhinitis Mouse Models

Mice were sensitized via intraperitoneal administration of 0.1 mg of ovalbumin (albumin from chicken egg white, Grade V, OVA, Sigma) and 1 mg of alum on Days 0, 7, 14, and 21. OVA was administered through the nasal cavity by inhalation in an amount of 0.2 mg/day successively from Days 21 to 27 to elicit symptoms. As the indicator for rhinitis symptoms, the number of times of sneezing for 5 minutes was counted immediately after the final elicitation on Day 27.

### Histopathological Examination

Auricle tissue samples were recovered from the models for contact hypersensitivity responses and fixed in 10% formalin to prepare paraffin blocks. The resultants were sliced and histopathologically examined via May Grunwald/Giemsa staining. Tissue slices obtained from the nasal mucous membrane of rhinitis models 12 hours after the final elicitation were histopathologically examined via May Grunwald/Giemsa staining. The infiltrated mononuclear cells, neutrophils, eosinophils, and mast cells were counted at 400-fold magnification in at least 5 fields and quantified.

### Statistical Analysis

A statistically significant difference was examined by the student's t-test.

### Results

1. Suppression of STAT6 Expression by Introduction of Human Stat6 siRNA

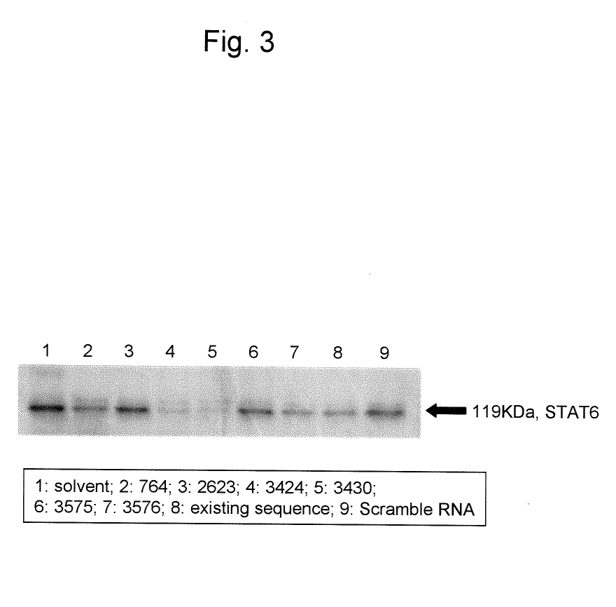

The 6 prepared candidate sequences for human STAT6 siRNA were subjected to Western blotting so as to determine a sequence capable of efficiently suppressing STAT6 expression. The results are shown in FIG. 3. In FIG. 3, lanes 2 to 7 represent the 6 types of STAT6 siRNAs that were newly prepared this time, and lane 8 represents an existing sequence (Rippmann J. F. et al., FEBS Lett., 2005, 579: 173-178). Among the newly prepared STAT6 siRNAs, those represented by lanes 4 and 5 show the effects of STAT6 expression suppression that are more potent than those attained by existing STAT6 siRNAs at a significant level.

Therefore, STAT6 siRNAs having such 2 sequences were to be used for the following experiment (hereafter, “3424” represents STAT6 siRNA 3 and “3430” represents STAT6 siRNA 4). The nucleotide sequences are as shown below.

3424 (STAT6 siRNA 3)
(SEQ ID NO: 5)
5′-GCUUCUGAUACGUGUAUGAGA sense strand
(SEQ ID NO: 6)
UCCGAAGACUAUGCACAUACU-5′ anti-sense strand
3430 (STAT6 siRNA 4)
(SEQ ID NO: 7)
5′-GAUACGUGUAUGAGACUAUGC sense strand
(SEQ ID NO: 8)
GACUAUGCACAUACUCUGAUA-5′ anti-sense strand

2. Production of Eotaxin-3 by Human Fibroblast into which Human STAT6 siRNA has been Introduced

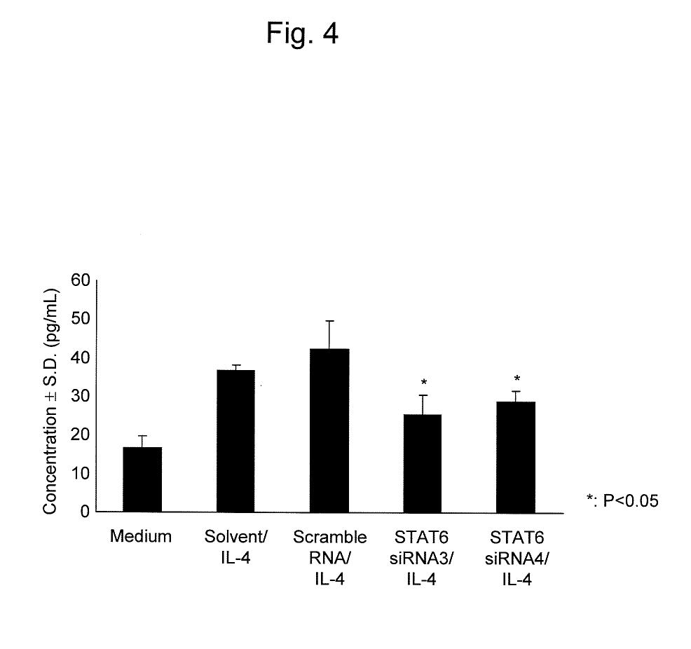

Upon IL-4 stimulation, production of eotaxin-3 by human fibroblasts depending on the STAT6 pathway was significantly suppressed via introduction of STAT6 siRNA 3 or STAT6 siRNA 4 selected in Experiment 1. FIG. 4 shows the results of ELISA assay. Without stimulation, production of eotaxin-3 from fibroblasts was not influenced (data not shown).

3. Suppression of STAT6 Expression in Human Fibroblasts into which Human Stat6 shRNA Had been Introduced

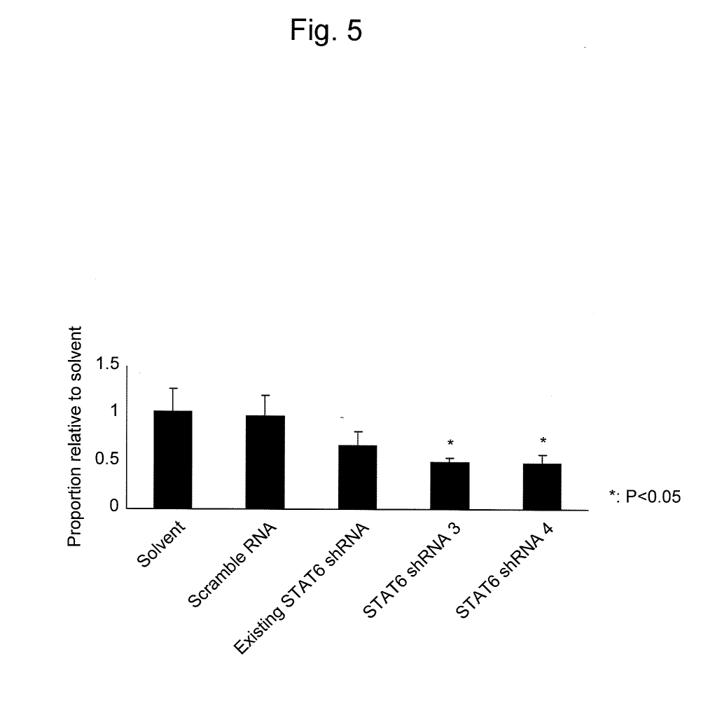

shRNAs were prepared based on the 2 siRNA sequences that had exhibited the effects in Experiments 1 and 2 (STAT6 shRNA3 and STAT6 shRNA4). The effects thereof on STAT6 expression in vitro were examined. As with the results regarding siRNA, two new sequences were found to produce more potent effects of suppressing STAT6 expression at the mRNA level via real-time PCR. FIG. 5 shows the results of ELISA assay.

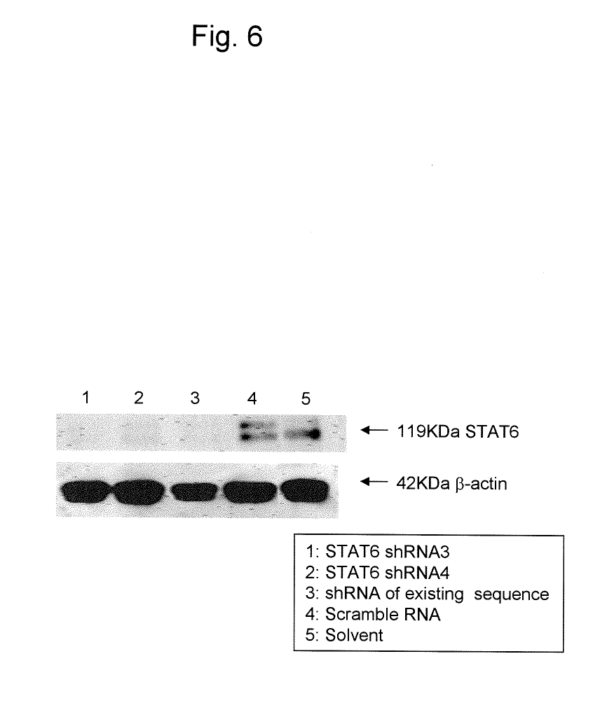

STAT6 expression at the protein level examined via Western blotting was found to be suppressed upon shRNA introduction. While shRNA prepared based on existing sequences shows the equivalent effects of suppressing STAT6 expression (lane 3 in FIG. 6), the newly prepared sequences are considered to have more potent suppression effects, in comparison with β-actin (FIG. 6).

4. Production of Eotaxin-3 in Human Fibroblasts into which Human Stat6 shRNA Had been Introduced

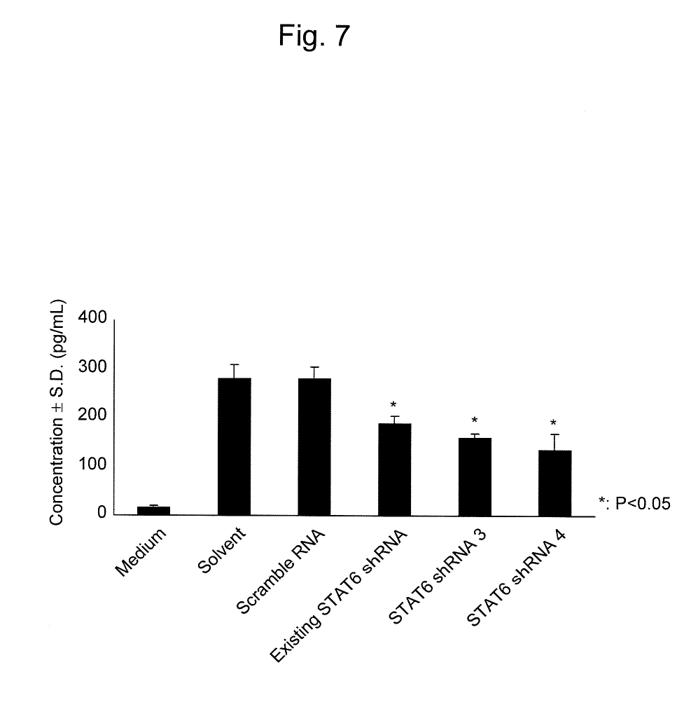

As with the case in which siRNA had been introduced, the newly prepared sequence exhibited more potent effects of suppressing eotaxin-3 expression than existing sequences upon shRNA introduction. FIG. 7 shows the results of ELISA assay.

5. Effects of Introduction of Mouse STAT6 siRNA on STAT6 Expression in Normal Fibroblasts

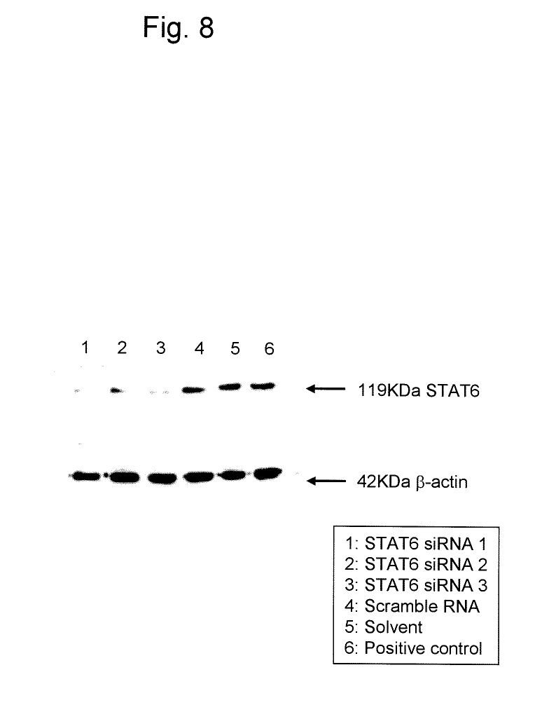

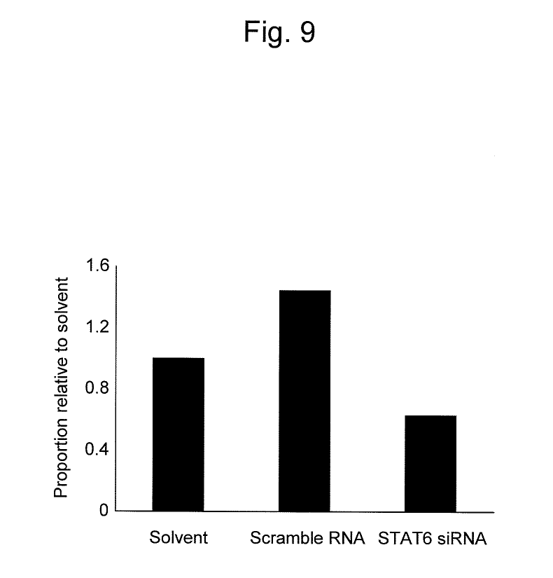

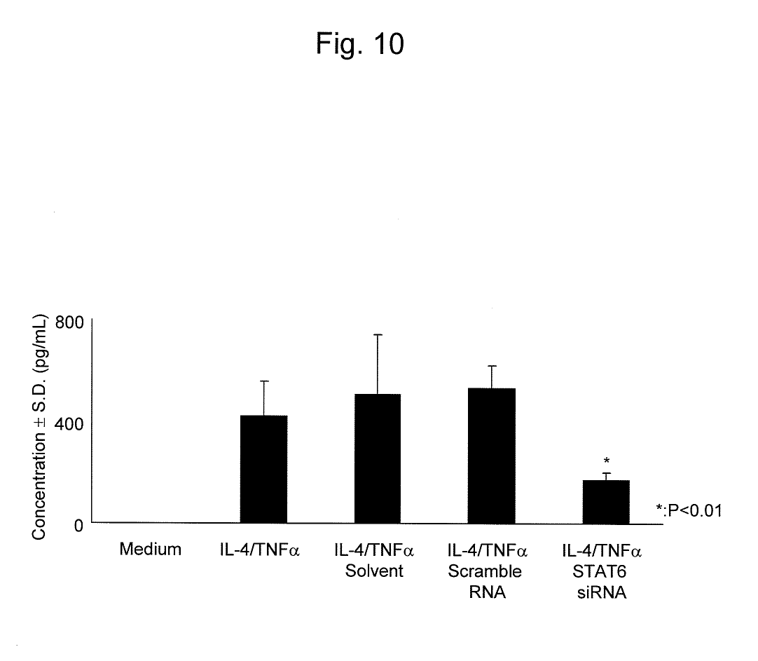

Candidate sequences for mouse STAT6 siRNA are shown below. FIG. 8 shows the results of suppression of STAT6 protein expression confirmed via Western blotting. As shown in FIG. 8, all of STAT6 siRNAs 1, 2, and 3 exhibited the effects of suppressing STAT6 protein expression. STAT6 siRNA 3 that had most efficiently suppressed STAT6 protein expression was used as STAT6 siRNA below. FIG. 9 shows the results of suppression of STAT6 mRNA expression confirmed via RT-PCR, and FIG. 10 shows the results of quantification via ELISA of the concentration of eotaxin (CCL11) produced upon IL-4 (10 ng/ml) and TNF-alpha (40 ng/ml) costimulation. As shown in FIG. 9 and FIG. 10, newly developed mouse STAT6 siRNA exhibited the effects of suppressing the expression of STAT6 mRNA and the production of eotaxin (CCL11).

Mouse STAT6 siRNA 1
(SEQ ID NO: 9)
5′-GCCGAGGCACCCUGUAUAUCC sense strand
(SEQ ID NO: 10)
GACGGCUCCGUGGGACAUAUA-5′ anti-sense strand
Mouse STAT6 siRNA 2
(SEQ ID NO: 11)
5′-CCUGGUUCUGUUAAGGAUUCA sense strand
(SEQ ID NO: 12)
GGGGACCAAGACAAUUCCUAA-5′ anti-sense strand
Mouse STAT6 siRNA3
(SEQ ID NO: 13)
5′-CGAAUGUGAUACAACUGUAUC sense strand
(SEQ ID NO: 14)
GAGCUUACACUAUGUUGACAU-5′ anti-sense strand

6. Effects of STAT6 siRNA on Contact Hypersensitivity Responses

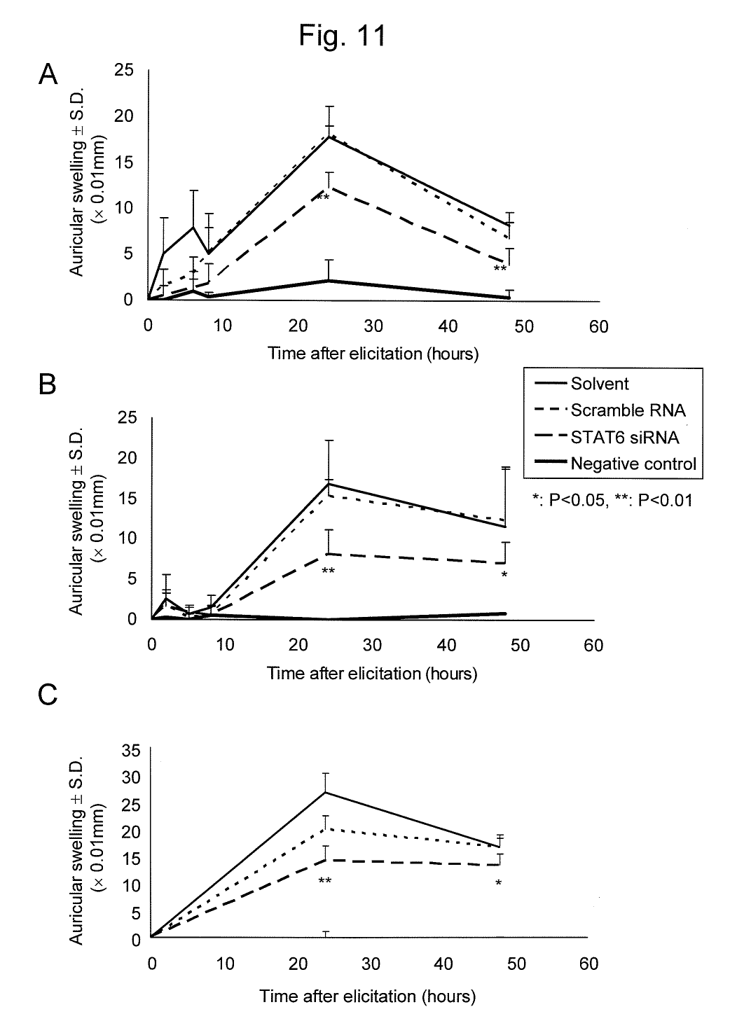

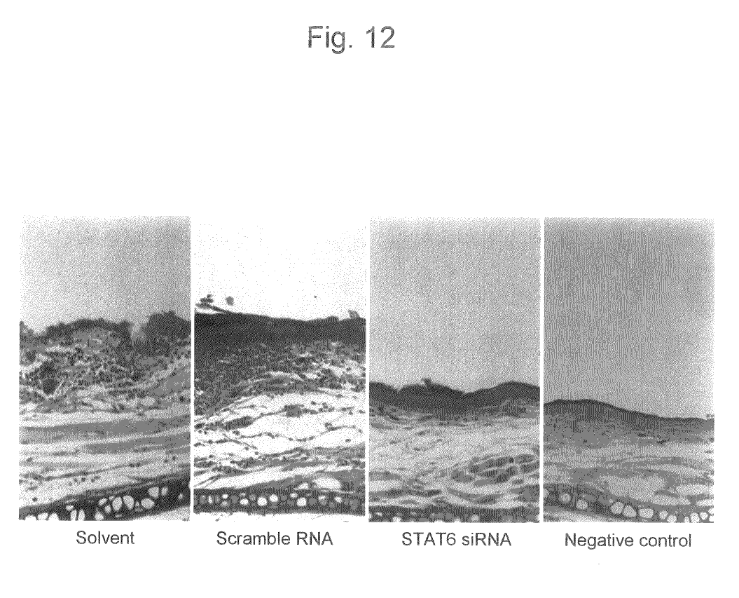

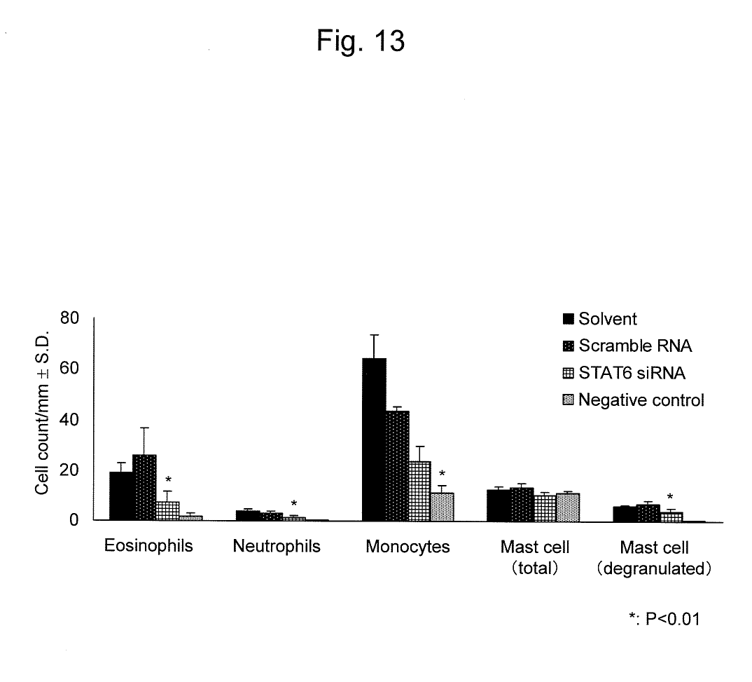

Contact hypersensitivity responses to TNCB, DNFB, and oxazolone were significantly suppressed by subcutaneous administration of STAT6 siRNA to the auricles (FIG. 11). Contact hypersensitivity responses to TNCB were histopathologically examined via Giemsa staining. As a result, significant reduction in edema and cellular infiltration was observed in the group to which STAT6 siRNA had been administered, in comparison with the control group (FIG. 12). As a result of examination of the profiles of infiltrated cells, the number of mononuclear cells, eosinophils, neutrophils, and degranulated mast cells was found to have decreased (FIG. 13).

7. Effects of STAT6 siRNA-Containing Ointment on Contact Hypersensitivity Responses

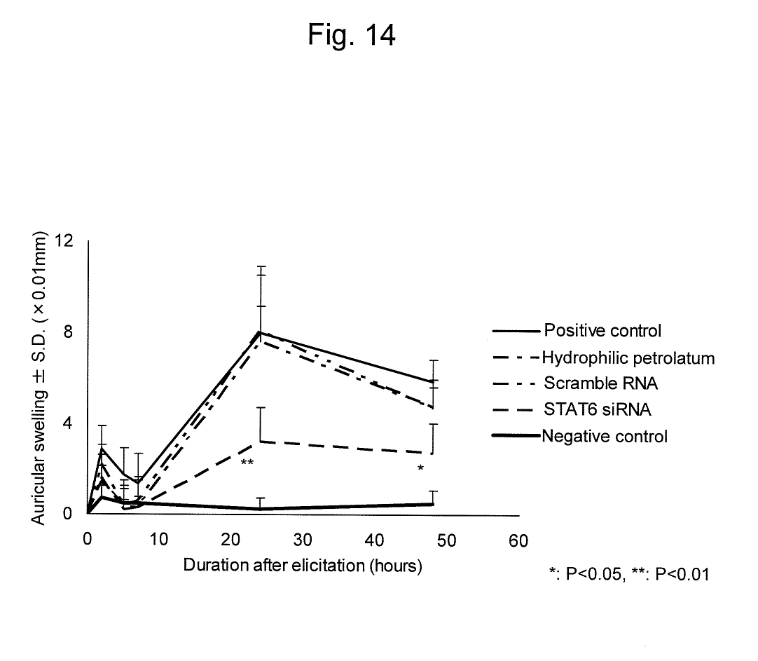

The STAT6 siRNA-containing ointment was prepared and applied to the auricles. As a result, auricular swelling was significantly suppressed in comparison with the control group (FIG. 14). Based on such results, use of STAT6 siRNA for an ointment was considered to be possible.

8. Effects of STAT6 siRNA on Rhinitis Models

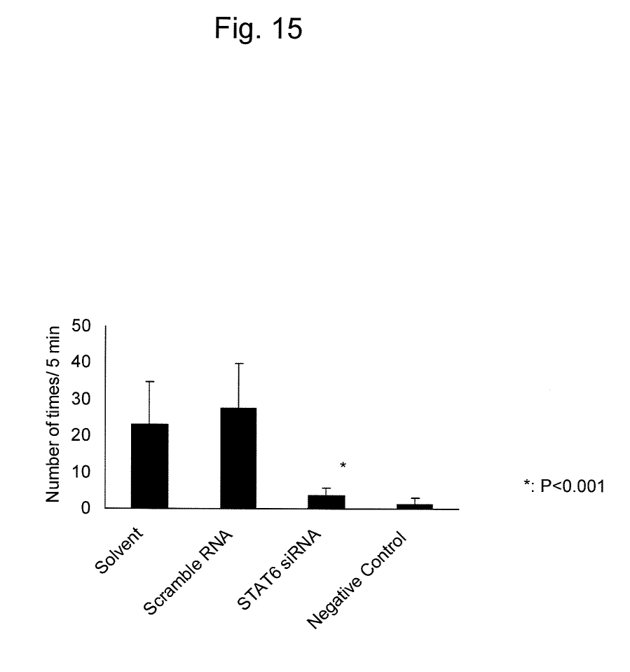

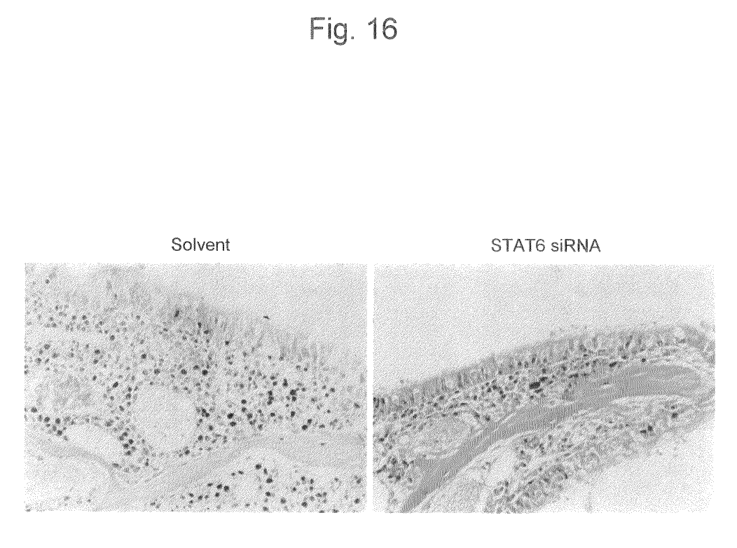

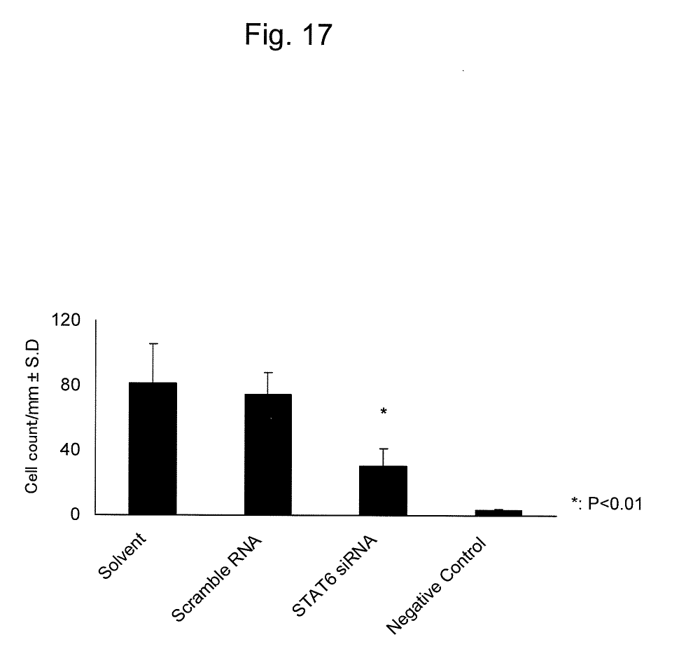

OVA was administered to the OVA-sensitized mice through the nasal cavity by inhalation successively from Day 21 to Day 27 to elicit rhinitis reactions. STAT6 siRNA dissolved in PBS was applied to the nasal cavity on Days 22, 23, and 24 (3 nmol/day), and the therapeutic effects thereof were examined. As a result, the number of times of sneezing was significantly decreased via administration of STAT6 siRNA (FIG. 15). Suppression of inflammatory reactions was histopathologically confirmed in terms of, for example, significant decrease in eosinophilic infiltration. FIG. 16 shows the results of Giemsa staining and FIG. 17 shows the number of infiltrated eosinophils.

### INDUSTRIAL APPLICABILITY

Use of the dsRNA molecule of the present invention enables specific suppression of STAT6 gene expression and, in turn, enables inhibition of Th2-type cytokine and chemokine production. Thus, allergic diseases can be prevented and treated.

All publications, patents, and patent applications cited herein are incorporated herein by reference in their entirety.

### SEQUENCE LISTING FREE TEXT

SEQ ID NOs: 5 to 14; synthetic sequences

1. A double-stranded RNA (dsRNA) molecule targeting mRNA of the STAT6 gene composed of (a) or (b) below:
(a) a sense strand identical to a sequence comprising 15 to 50 continuous nucleotides of the nucleotide sequence resulting from substitution of thymine with uracil in the STAT6 gene sequence represented by SEQ ID NO: 1 or 3 and an antisense strand comprising a nucleotide sequence complementary to the nucleotide sequence of the sense strand; or
(b) a sense strand comprising a sequence derived from the sequence comprising at least 15 to 50 continuous nucleotides of the nucleotide sequence resulting from substitution of thymine with uracil in the STAT6 gene sequence represented by SEQ ID NO: 1 or 3 by deletion, substitution, or addition of 1 or several nucleotides and capable of hybridizing to the STAT6 gene and an antisense strand comprising a nucleotide sequence complementary to the nucleotide sequence of the sense strand.

2. The dsRNA molecule according to claim 1 composed of (c) or (d) below:
(c) a sense strand identical to a nucleotide sequence selected from the group consisting of: a sequence comprising nucleotides 3426 to 3446 of the nucleotide sequence resulting from substitution of thymine with uracil in the human STAT6 gene sequence represented by SEQ ID NO: 1; a sequence comprising nucleotides 3432 to 3452 of the nucleotide sequence resulting from substitution of thymine with uracil in the human STAT6 gene sequence represented by SEQ ID NO: 1; a sequence comprising nucleotides 259 to 279 of the nucleotide sequence resulting from substitution of thymine with uracil in the mouse STAT6 gene sequence represented by SEQ ID NO: 3; and a sequence comprising nucleotides 3026 to 3046 of the nucleotide sequence resulting from substitution of thymine with uracil in the mouse STAT6 gene sequence represented by SEQ ID NO: 3 and an antisense strand comprising a nucleotide sequence complementary to the nucleotide sequence of the sense strand; or
(d) a sense strand comprising a sequence derived from any nucleotide sequence selected from the group consisting of: a sequence comprising nucleotides 3426 to 3446 of the nucleotide sequence resulting from substitution of thymine with uracil in the human STAT6 gene sequence represented by SEQ ID NO: 1; a sequence comprising nucleotides 3432 to 3452 of the nucleotide sequence resulting from substitution of thymine with uracil in the human STAT6 gene sequence represented by SEQ ID NO: 1; a sequence comprising nucleotides 259 to 279 of the nucleotide sequence resulting from substitution of thymine with uracil in the mouse STAT6 gene sequence represented by SEQ ID NO: 3; and a sequence comprising nucleotides 3026 to 3046 of the nucleotide sequence resulting from substitution of thymine with uracil in the mouse STAT6 gene sequence represented by SEQ ID NO: 3 by deletion, substitution, or addition of 1 or several nucleotides and capable of hybridizing to the STAT6 gene sequence and an antisense strand comprising a nucleotide sequence complementary to the nucleotide sequence of the sense strand.

3. The dsRNA molecule according to claim 2, which is composed of any base pairs selected from the group consisting of base pairs comprising a sense strand represented by SEQ ID NO: 5 and an antisense strand represented by SEQ ID NO: 6; base pairs comprising a sense strand represented by SEQ ID NO: 7 and an antisense strand represented by SEQ ID NO: 8; base pairs comprising a sense strand represented by SEQ ID NO: 9 and an antisense strand represented by SEQ ID NO: 10; base pairs comprising a sense strand represented by SEQ ID NO: 11 and an antisense strand represented by SEQ ID NO: 12; and base pairs comprising a sense strand represented by SEQ ID NO: 13 and an antisense strand represented by SEQ ID NO: 14.

4. The dsRNA molecule according to claim 1, wherein the sense strand is ligated to the antisense strand via a linker molecule.

5. A vector comprising template DNA of the dsRNA molecule according to claim 4, wherein the vector expresses the dsRNA molecule.

6. (canceled)

7. (canceled)

8. The dsRNA molecule according to claim 1, wherein the dsRNA molecule has an overhanging nucleotide comprising one or a plurality of guanines (Gs) at the 5′ end of the sense strand.

9. The dsRNA molecule according to claim 8, wherein the sense strand is ligated to the antisense strand via a linker molecule.

10. A vector comprising template DNA of the dsRNA molecule according to claim 9, wherein the vector expresses the dsRNA molecule.

11-14. (canceled)

15. A composition capable of suppressing STAT6 gene expression without inducing interferon or cell damage comprising a pharmaceutically acceptable carrier and at least one dsRNA molecule selected from the group consisting of the dsRNA molecule of claim 1; the dsRNA molecule of claim 1, wherein the sense strand is ligated to the antisense strand via a linker molecule; the dsRNA molecule of claim 1, wherein the dsRNA molecule has an overhanging nucleotide comprising one or a plurality of guanines at the 5′ end of the sense strand; and the dsRNA molecule of claim 1, wherein the dsRNA molecule has an overhanging nucleotide comprising one or a plurality of guanines at the 5′ end of the sense strand and wherein the sense strand is ligated to the antisense strand via a linker molecule.

16. A composition capable of suppressing STAT6 gene expression without inducing interferon or cell damage comprising a pharmaceutically acceptable carrier and a vector comprising template DNA of at least one dsRNA molecule selected from the group consisting of the dsRNA molecule of claim 1, wherein the sense strand is ligated to the antisense strand via a linker molecule; and the dsRNA molecule of claim 1, wherein the dsRNA molecule has an overhanging nucleotide comprising one or a plurality of guanines at the 5′ end of the sense strand and wherein the sense strand is ligated to the antisense strand via a linker molecule.

17. A method for treating or preventing an allergic disorder comprising administering the pharmaceutical composition of claim 15 to a mammalian subject in need thereof.

18. The method of claim 17, wherein administering the pharmaceutical composition comprises administering the pharmaceutical composition through the nasal cavity or applying the pharmaceutical composition to the skin.

19. The method of claim 17, wherein the allergic disorder is selected from the group consisting of rhinostenosis, allergic bronchitis, allergic conjunctivitis, inflammatory disease, rash, hives, atopic dermatitis, allergic rhinitis, allergic conjunctivitis, allergic gastro enteritis, bronchial asthma, pediatric asthma, alimentary allergy, and drug allergy.

20. A method for treating or preventing an allergic disorder comprising administering the pharmaceutical composition of claim 16 to a mammalian subject in need thereof.

21. The method of claim 20, wherein administering the pharmaceutical composition comprises administering the pharmaceutical composition through the nasal cavity or applying the pharmaceutical composition to the skin.

22. The method of claim 20, wherein the allergic disorder is selected from the group consisting of rhinostenosis, allergic bronchitis, allergic conjunctivitis, inflammatory disease, rash, hives, atopic dermatitis, allergic rhinitis, allergic conjunctivitis, allergic gastro enteritis, bronchial asthma, pediatric asthma, alimentary allergy, and drug allergy.


SEQUENCE LISTING (14 sequences)

SEQ ID NO: 1 - DNA, 3993 bp, Homo sapiens
  CDS (272)..(2815)
ccggaaacagcgggctggggcagccactgcttacactgaagagggaggacgggagaggagtgtgtgtgtgtgtgtgtgtgtgtgtgtgtatgtatgtgtgtgctttatcttatttttctttttggtggtggtggtggaaggggggaggtgctagcagggccagccttgaactcgctggacagagctacagacctatggggcctggaagtgcccgctgagaaagggagaagacagcagaggggttgccgaggcaacctccaagtcccagatcatgtctctgtggggtctggtctccaagatgcccccagaaaaagtgcagcggctctatgtcgactttccccaacacctgcggcatcttctgggtgactggctggagagccagccctgggagttcctggtcggctccgacgccttctgctgcaacttggctagtgccctactttcagacactgtccagcaccttcaggcctcggtgggagagcagggggaggggagcaccatcttgcaacacatcagcacccttgagagcatatatcagagggaccccctgaagctggtggccactttcagacaaatacttcaaggagagaaaaaagctgttatggaacagttccgccacttgccaatgcctttccactggaagcaggaagaactcaagtttaagacaggcttgcggaggctgcagcaccgagtaggggagatccaccttctccgagaagccctgcagaagggggctgaggctggccaagtgtctctgcacagcttgatagaaactcctgctaatgggactgggccaagtgaggccctggccatgctactgcaggagaccactggagagctagaggcagccaaagccctagtgctgaagaggatccagatttggaaacggcagcagcagctggcagggaatggcgcaccgtttgaggagagcctggccccactccaggagaggtgtgaaagcctggtggacatttattcccagctacagcaggaggtaggggcggctggtggggagcttgagcccaagacccgggcatcgctgactggccggctggatgaagtcctgagaaccctcgtcaccagttgcttcctggtggagaagcagcccccccaggtactgaagactcagaccaagttccaggctggagttcgattcctgttgggcttgaggttcctgggggccccagccaagcctccgctggtcagggccgacatggtgacagagaagcaggcgcgggagctgagtgtgcctcagggtcctggggctggagcagaaagcactggagaaatcatcaacaacactgtgcccttggagaacagcattcctgggaactgctgctctgccctgttcaagaacctgcttctcaagaagatcaagcggtgtgagcggaagggcactgagtctgtcacagaggagaagtgcgctgtgctcttctctgccagcttcacacttggccccggcaaactccccatccagctccaggccctgtctctgcccctggtggtcatcgtccatggcaaccaagacaacaatgccaaagccactatcctgtgggacaatgccttctctgagatggaccgcgtgccctttgtggtggctgagcgggtgccctgggagaagatgtgtgaaactctgaacctgaagttcatggctgaggtggggaccaaccgggggctgctcccagagcacttcctcttcctggcccagaagatcttcaatgacaacagcctcagtatggaggccttccagcaccgttctgtgtcctggtcgcagttcaacaaggagatcctgctgggccgtggcttcaccttttggcagtggtttgatggtgtcctggacctcaccaaacgctgtctccggagctactggtctgaccggctgatcattggcttcatcagcaaacagtacgttactagccttcttctcaatgagcccgacggaacctttctcctccgcttcagcgactcagagattgggggcatcaccattgcccatgtcatccggggccaggatggctctccacagatagagaacatccagccattctctgccaaagacctgtccattcgctcactgggggaccgaatccgggatcttgctcagctcaaaaatctctatcccaagaagcccaaggatgaggctttccggagccactacaagcctgaacagatgggtaaggatggcaggggttatgtcccagctaccatcaagatgaccgtggaaagggaccaaccacttcctaccccagagctccagatgcctaccatggtgccttcttatgaccttggaatggcccctgattcctccatgagcatgcagcttggcccagatatggtgccccaggtgtacccaccacactctcactccatccccccgtatcaaggcctctccccagaagaatcagtcaacgtgttgtcagccttccaggagcctcacctgcagatgccccccagcctgggccagatgagcctgccctttgaccagcctcacccccagggcctgctgccgtgccagcctcaggagcatgctgtgtccagccctgaccccctgctctgctcagatgtgaccatggtggaagacagctgcctgagccagccagtgacagcgtttcctcagggcacttggattggtgaagacatattccctcctctgctgcctcccactgaacaggacctcactaagcttctcctggaggggcaaggggagtcggggggagggtccttgggggcacagcccctcctgcagccctcccactatgggcaatctgggatctcaatgtcccacatggacctaagggccaaccccagttggtgatcccagctggagggagaacccaaagagacagctcttctactacccccacagacctgctctggacacttgctcatgccctgccaagcagcagatggggagggtgccctcctatccccacctactcctgggtcaggaggaaaagactaacaggagaatgcacagtgggtggagccaatccactccttcctttctatcattcccctgcccacctccttccagcactgactggaagggaagttcaggctctgagacacgccccaacatgcctgcacctgcagcgcgcacacgcacgcacacacacatacagagctctctgagggtgatggggctgagcaggaggggggctgggtaagagcacaggttagggcatggaaggcttctccgcccattctgacccagggcctaggacggataggcaggaacatacagacacatttacactagaggccagggatagaggatattgggtctcagccctaggggaatgggaagcagctcaagggaccctgggtgggagcataggaggggtctggacatgtggttactagtacaggttttgccctgattaaaaaatctcccaaagccccaaattcctgttagccaggtggaggcttctgatacgtgtatgagactatgcaaaagtacaagggctgagattcttcgtgtatagctgtgtgaacgtgtatgtacctaggatatgttaaatgtatagctggcaccttagttgcatgaccacatagaacatgtgtctatctgcttttgcctacgtgacaacacaaatttgggagggtgagacactgcacagaagacagcagcaagtgtgctggcctctctgacatatgctaacccccaaatactctgaatttggagtctgactgtgcccaagtgggtccaagtggctgtgacatctacgtatggctccacacctccaatgctgcctgggagccagggtgagagtctgggtccaggcctggccatgtggccctccagtgtatgagagggccctgcctgctgcatcttttctgttgccccatccaccgccagcttcccttcactcccctatcccattctccctctcaaggcaggggtcatagatcctaagccataaaataaattttattccaaaataacaaaataaataatctactgtacacaatctgaaaaaaaaaaaaaaaaaa

SEQ ID NO: 2 - protein, 847 aa, Homo sapiens
MSLWGLVSKMPPEKVQRLYVDFPQHLRHLLGDWLESQPWEFLVGSDAFCCNLASALLSDTVQHLQASVGEQGEGSTILQHISTLESIYQRDPLKLVATFRQILQGEKKAVMEQFRHLPMPFHWKQEELKFKTGLRRLQHRVGEIHLLREALQKGAEAGQVSLHSLIETPANGTGPSEALAMLLQETTGELEAAKALVLKRIQIWKRQQQLAGNGAPFEESLAPLQERCESLVDIYSQLQQEVGAAGGELEPKTRASLTGRLDEVLRTLVTSCFLVEKQPPQVLKTQTKFQAGVRFLLGLRFLGAPAKPPLVRADMVTEKQARELSVPQGPGAGAESTGEIINNTVPLENSIPGNCCSALFKNLLLKKIKRCERKGTESVTEEKCAVLFSASFTLGPGKLPIQLQALSLPLVVIVHGNQDNNAKATILWDNAFSEMDRVPFVVAERVPWEKMCETLNLKFMAEVGTNRGLLPEHFLFLAQKIFNDNSLSMEAFQHRSVSWSQFNKEILLGRGFTFWQWFDGVLDLTKRCLRSYWSDRLIIGFISKQYVTSLLLNEPDGTFLLRFSDSEIGGITIAHVIRGQDGSPQIENIQPFSAKDLSIRSLGDRIRDLAQLKNLYPKKPKDEAFRSHYKPEQMGKDGRGYVPATIKMTVERDQPLPTPELQMPTMVPSYDLGMAPDSSMSMQLGPDMVPQVYPPHSHSIPPYQGLSPEESVNVLSAFQEPHLQMPPSLGQMSLPFDQPHPQGLLPCQPQEHAVSSPDPLLCSDVTMVEDSCLSQPVTAFPQGTWIGEDIFPPLLPPTEQDLTKLLLEGQGESGGGSLGAQPLLQPSHYGQSGISMSHMDLRANPSW

SEQ ID NO: 3 - DNA, 3805 bp, Mus musculus
  CDS (286)..(2799)
gagaagccggaaacagcaggccggggcagccagggtttacagtgaagaaggcccggagacgagtgcgtgcgtgcctgtgtgtgtgtttgtgtgtgtgtgcgcgcgctcgagcgtgtgcgcgcgtgcctgtgtgcatatgtgtgtgtgtgtctgtacacattgagtttttagggccagcccaggacccgctggacagacctacagacccatggggcttggtagtgccctctgagagagggagaagatagcagcggggctgctgaggcaccctgtatatcccagatcatgtctctgtggggcctaatttccaagatgtccccagaaaaactgcaacggctctatgttgactttccacaacgcctacggcatctcctggctgactggctggagagccagccctgggagttcctggtcggttcagatgctttctgttacaacatggccagtgccctactttctgccacagtccagcgtcttcaggccactgctggagagcaggggaagggaaacagcatcttgccgcacatcagcaccttggagagcatctatcagagggaccccctgaagctggtggccaccatcagacaaatacttcaaggggagaaaaaagctgttatagaagagttccgccacctgccagggcccttccatcggaagcaggaagaactcaagtttactacagccctcggaaggcttcagcatcgagtaagggagacccggcttctccgagaatctctgcagcaaggggctaagactggacaagtgtctctgcagaatttgatagaccctcctgtcaatggtcctggtccaagtgaggacctggccaccatgctgcaggggactgtgggggacctggaggccacccaggccctggtgctgaaaaggattcagatttggaagcggcagcaacagctggcagggaatggcacaccctttgaggagagcctagcagggctgcaggagaggtgtgaaagcctggtggaaatttattcccagctgcagcaggagattggggcagccagtggagaactggaacccaagacccgggcatcgctgataagccgtctggatgaagtcctgcgaacccttgtgaccagctctttcctggtggagaagcagcccccgcaggttctgaagacacagactaagttccaggctggggttcgattcctgctgggtctgcagtttctagggacctcagccaagcctccaatggtcagagctgacatggtgacagagaaacaggccagagaactaagcctggcccaggggcccgggactggagtggagagcacaggagagatcatgaacaacacggtgcccctggagaacagcattcccagcaactgctgctccgccctgttcaagaacctgctcctgaagaaaataaagcgctgtgagcggaagggcacagagtctgtcaccgaggagaagtgtgctgtgctcttctccacgagcttcacattgggccccaacaaacttctcatccagcttcaggccctgtctctgcccttggtggtcatcgtgcatggtaaccaagacaacaacgccaaagctaccatcctatgggacaatgccttctctgagatggaccgagtgccctttgtggtggctgagcgagtgccctgggagaagatgtgtgaaaccctaaacctcaagtttatggctgaggtggggaccagccggggactgcttccagagcacttcctgttcctcgcccagaagatcttcaacgacaacagcctcagtgtggaggcctttcagcaccgctgtgtgtcctggtcacagttcaataaggagatcctgctgggccgaggcttcacattttggcagtggtttgatggtgtcctggacctcaccaaacgctgtctccggagctactggtcagatcggctgatcattggctttattagtaagcaatatgtcactagccttctcctcaatgagccagatgggaccttcctcctccgctttagcgactctgagatcgggggcatcaccattgcacacgtcatccggggtcaggatggctcctcacagatagagaacatccagccattttctgccaaagacctgtccattcgctcactgggggaccggatccgggatcttgctcagttaaaaaacctctaccccaagaaacccaaagatgaggctttccggagtcactataagcccgaacagatggggaaggacgggaggggttatgtctctactactatcaagatgactgtggaaagggaccagccccttcctactccagagccccagatgcctgccatggtgccaccttatgatcttggaatggcccctgatgcttccatgcaactcagctcagatatggggtatcctccacagtccatccactcatttcagagcctagaagagtccatgagtgtactgccatcttttcaggagcctcacctgcaaatgccccccaacatgagccagataaccatgccctttgaccagcctcacccccagggtctgctgcagtgccagtcccaggaacatgctgtgtccagccctgaacccatgctttgctcagatgtgactatggtagaggacagctgcctaactcagcctgtgggaggtttcccccaaggcacctgggtcagtgaagacatgtaccctcccctgatgcctcccactgaacaggacctcaccaagcttctcctggagaaccaaggggaggcaggagggtccttaggaagccagcccctcctgcaaccatctccttatgggcaatcagggatctcactgtcccacctggacctaaggaccaaccccagctggtgatcccagctggagaagcccagaaacaaagcctcttctgtctctatggaccagctctggacacctgctcatgcaggtgccttccgtctcaactgttccttggtcaagagaaaagaactggctgggagaccatgtggtgtatggaactgctgtgctctgtccttcctgccatatcagggccccccttttccagcactgggtgcaaagggatgagtggggtgttaatgctcgaatgtgatacaactgtatcacaacacacacgcacacacacatacacacacaccagaactgtgttgagccagggcctgggactcaacatacagaaacatagagacattgtgccaaagacagagggcatataggcctagggcattgaagctgggctcaggtgactctgggagggagaaaaaggaaaaagtgggtatacagtcactggtgtgagttctacccagattttaaaaaacaagactccaaagctccaaattcttgcaaaaaaagatgcctagtgacatttgagactgcattctaagagctaagcttgtgtatagctgtaccaatgtttacccaagacatgttaacctatagaagtcacacatcactgtatgaccgcacagaacatgtatcttctgcttttgccagtgtgaccttaacatatctgaaaggctgagacattgtataagacagcaacccagtatcatttggggagtaactatgtggctgtgacatgcataaagctctagcctgggtaacttgatgcttccagtgtttcctagagcctgggatataagtagggatgcagacctctctgtgtaaactccctgggcgttgaggccctcaactgctgtctcttgtacttttctgtccactgccagctctgcccccctcccccggctcttacccagtcctttttccctcactggaggggaagggggccatggatcctaagccataaaataaattttattccaaaataacaaaataaataatctactgtacacaatctgaaaag

SEQ ID NO: 4 - protein, 837 aa, Mus musculus
MSLWGLISKMSPEKLQRLYVDFPQRLRHLLADWLESQPWEFLVGSDAFCYNMASALLSATVQRLQATAGEQGKGNSILPHISTLESIYQRDPLKLVATIRQILQGEKKAVIEEFRHLPGPFHRKQEELKFTTALGRLQHRVRETRLLRESLQQGAKTGQVSLQNLIDPPVNGPGPSEDLATMLQGTVGDLEATQALVLKRIQIWKRQQQLAGNGTPFEESLAGLQERCESLVEIYSQLQQEIGAASGELEPKTRASLISRLDEVLRTLVTSSFLVEKQPPQVLKTQTKFQAGVRFLLGLQFLGTSAKPPMVRADMVTEKQARELSLAQGPGTGVESTGEIMNNTVPLENSIPSNCCSALFKNLLLKKIKRCERKGTESVTEEKCAVLFSTSFTLGPNKLLIQLQALSLPLVVIVHGNQDNNAKATILWDNAFSEMDRVPFVVAERVPWEKMCETLNLKFMAEVGTSRGLLPEHFLFLAQKIFNDNSLSVEAFQHRCVSWSQFNKEILLGRGFTFWQWFDGVLDLTKRCLRSYWSDRLIIGFISKQYVTSLLLNEPDGTFLLRFSDSEIGGITIAHVIRGQDGSSQIENIQPFSAKDLSIRSLGDRIRDLAQLKNLYPKKPKDEAFRSHYKPEQMGKDGRGYVSTTIKMTVERDQPLPTPEPQMPAMVPPYDLGMAPDASMQLSSDMGYPPQSIHSFQSLEESMSVLPSFQEPHLQMPPNMSQITMPFDQPHPQGLLQCQSQEHAVSSPEPMLCSDVTMVEDSCLTQPVGGFPQGTWVSEDMYPPLMPPTEQDLTKLLLENQGEAGGSLGSQPLLQPSPYGQSGISLSHLDLRTNPSW

SEQ ID NO: 5 - RNA, 21 nt, Artificial
  Synthetic
gcuucugauacguguaugaga

SEQ ID NO: 6 - RNA, 21 nt, Artificial
  Synthetic
uccgaagacuaugcacauacu

SEQ ID NO: 7 - RNA, 21 nt, Artificial
  Synthetic
gauacguguaugagacuaugc

SEQ ID NO: 8 - RNA, 21 nt, Artificial
  Synthetic
gacuaugcacauacucugaua

SEQ ID NO: 9 - RNA, 21 nt, Artificial
  Synthetic
gccgaggcacccuguauaucc

SEQ ID NO: 10 - RNA, 21 nt, Artificial
  Synthetic
gacggcuccgugggacauaua

SEQ ID NO: 11 - RNA, 21 nt, Artificial
  Synthetic
ccugguucuguuaaggauuca

SEQ ID NO: 12 - RNA, 21 nt, Artificial
  Synthetic
ggggaccaagacaauuccuaa

SEQ ID NO: 13 - RNA, 21 nt, Artificial
  Synthetic
cgaaugugauacaacuguauc

SEQ ID NO: 14 - RNA, 21 nt, Artificial
  Synthetic
gagcuuacacuauguugacau